(19) United States
(12) Patent Application Publication
(10) Pub. No.: US 2009/0155357 A1
(43) Pub. Date: Jun. 18, 2009

(54) Alcohol Resistant Pharmaceutical Formulations

(75) Inventor: Goutam Muhuri, Belle Mead, NJ (US)

Correspondence Address: MCDONNELL BOEHNEN HULBERT & BERGHOFF LLP, 300 S. WACKER DRIVE, 32ND FLOOR, CHICAGO, IL, 60606, US

(73) Assignee: ALPHARMA INC., Fort Lee, NJ (US)

(21) Appl. No.: 11/995,184

(22) PCT Filed: Jul. 31, 2006
(86) PCT No.: PCT/US06/29932

§ 371 Date: Jul. 23, 2008

Related U.S. Application Data
(60) Provisional application No. 60/704,514, filed on Aug. 1, 2005.

(51) Int. Cl. A61K 9/28; A61K 9/52

(52) U.S. Cl. 424/457; 424/468

(57) ABSTRACT

The present invention provides alcohol resistant oral dosage pharmaceutical forms and methods of using such oral dosage forms to avoid dose dumping if the dosage form is taken together with alcohol.

### CROSS REFERENCE

This application claims the priority of U.S. Provisional Patent Application Ser. No. 60/704,514, which is incorporated by reference herein in its entirety.

### BACKGROUND OF THE INVENTION

There is concern that the modified or extended release characteristics of some pharmaceutical forms could be compromised in the presence of alcohol, which could lead to a “dosage dump” of a drug that is intended for administration in a non-immediate release fashion. Thus, there is a need in the art for modified release pharmaceutical forms that are not compromised in the presence of alcohol.

### SUMMARY OF THE INVENTION

In one aspect, the present invention provides modified release oral dosage forms, comprising

(a) a therapeutic agent and;

(b) an alcohol insoluble coating, wherein between 0% and 35% of the therapeutic agent is released from the dosage form in vitro after 60 minutes in the presence of 40% alcohol at pH 1.2.

In a further embodiment, the modified release comprises extended release or delayed release. In further embodiments, the alcohol insoluble coating is water insoluble. In various further embodiments, the alcohol insoluble coating comprises one or more compounds listed in Table 1. In further embodiments, the alcohol insoluble coating comprises a 1% to 40% weight gain in the oral dosage form. In further embodiments, the alcohol insoluble coating is between 5 microns thick and 1000 microns thick.

In a further embodiment, the therapeutic comprises an analgesic, or a pharmaceutically acceptable salt thereof; such as an opioid analgesic, or a pharmaceutically acceptable salt thereof. In a further embodiment, the therapeutic further comprises a non-opioid drug.

In another aspect, the present invention provides methods for alleviating pain, comprising administering to an individual in need thereof an amount effective to alleviate pain of the oral dosage form comprising an analgesic therapeutic disclosed above. In a further embodiment, the individual in need thereof drinks alcoholic beverages or is otherwise exposed to alcohol.

### DETAILED DESCRIPTION OF THE INVENTION

The present invention provides alcohol resistant oral dosage pharmaceutical forms and methods of using such oral dosage forms to avoid dose dumping if the dosage form is taken together with alcohol. As used herein, “taken together with alcohol” includes simultaneously taking the oral dosage form and alcohol, as well as ingesting alcohol 0-2 hours, preferably 0-1 hour, before or after taking the oral dosage form.

Thus, in one aspect, the present invention provides a modified release oral dosage form, comprising or consisting of a therapeutic agent and an alcohol insoluble coating, wherein between 0% and 35% of the therapeutic agent is released from the dosage form in vitro after 60 minutes in the presence of 40% alcohol at pH 1.2. In more preferred embodiments, the invention provides a modified release oral dosage form, comprising or consisting of a therapeutic agent and an alcohol insoluble coating, wherein between 0% and 30%, between 0% and 25%, between 0% and 20%, between 0% and 18%, between 0% and 16%, between 0% and 15%, between 0% and 14%, between 0% and 13%, between 0% and 12%, between 0% and 11%, between 0% and 10%, between 0% and 9%, between 0% and 8%, between 0% and 7%, between 0% and 6%, between 0% and 5%, between 0% and 4%, between 0% and 3%, between 0% and 2%, or between 0% and 1% of the therapeutic agent is released from the dosage form in vitro after 60 minutes in the presence of 40% alcohol at pH 1.2.

As used herein, the term “modified release” includes any dosage form having drug release features based on time, course, and/or location that are designed to accomplish therapeutic or convenience objectives not offered by immediate release forms. Included within “modified release” dosage forms are “extended release” (allows a reduction in dosing frequency relative to immediate release) and “delayed release” (designed to release the therapeutic agent from the dosage form at a time other than promptly after administration). In one embodiment, the oral dosage form provides for delayed release of the therapeutic agent, until after transit of the dosage form through the stomach. In a further embodiment, the oral dosage form also comprises an extended release component, wherein the therapeutic agent is not released until after transit of the dosage form through the stomach, and then is released in an extended manner, at a desired rate.

As used herein, the term “alcohol insoluble coating” is any type of layering or coating of the oral dosage form that inhibits release of the therapeutic agent from the dosage form in the presence of alcohol as described herein, and which provides for modified release of the therapeutic agent. In a preferred embodiment, the alcohol insoluble coating is also water insoluble.

Non-limiting examples of alcohol insoluble coatings and water insoluble coatings are provided in Table 1, and include combinations of such coatings, or combinations of such coatings with other pharmaceutically acceptable agents. This Table provides guidance with respect to application and the ultimate dosage form using these specific embodiments (for example, layering in combination with binders and/or other ingredients; enteric coating alone or with binder and/or other ingredients). Especially preferred embodiments of the alcohol insoluble coatings are cellulose acetate phthalate (CAP), hydroxypropyl methylcellulose phthalate (HPMCP), ethyl cellulose with less than 46.5% ethoxyl group, wax, or combinations thereof.

The oral dosage forms of the invention are solid dosage forms and include, but are not limited to, tablets, capsules (for example, hard gel or soft gel capsules where one or more of the components of the capsule shell is an alcohol insoluble coating), beads, granules, microspheres, spheroids, and osmotic push pull systems (used as a drug delivery technology with one or more alcohol insoluble polymers used to coat the delivery device; see, for example, U.S. Pat. No. 6,284,274).

It will be recognized by one of skill in the art that the weight percent of the alcohol insoluble coating of the oral dosage compositions of the invention will vary dependent upon a number of factors, including, but not limited to, the solubility of the drug, the type of dosage form (i.e. tablet, pellet, etc.), and the specific composition of the alcohol insoluble coating, including any pharmacologically inactive ingredients, additional polymers, etc. In a preferred embodiment, for a tablet form, the alcohol insoluble coating comprises a 1% to 40% weight gain. In more preferred embodiments, for a tablet form, the alcohol insoluble coating comprises a 1% to 30% weight gain, a 1% to 25% weight gain, a 1% to 20% weight gain, a 1% to 15% weight gain, a 1% to 10% weight gain, a 1% to 5% weight gain, or a 1% to 3% weight gain. In a preferred embodiment, for a pellet form, the alcohol insoluble coating comprises a 1% to 95% weight gain. In more preferred embodiments, for a pellet form, the alcohol insoluble coating comprises a 5% to 95% weight gain, a 5% to 90% weight gain, a 5% to 80% weight gain, a 5% to 70% weight gain, a 5% to 60% weight gain, a 5% to 50% weight gain, a 5% to 40% weight gain, a 5% to 30% weight gain, or a 5% to 10% weight gain.

It will be recognized by one of skill in the art that the thickness of the alcohol insoluble coating will vary dependent upon a number of factors, including, but not limited to, the weight percent of the alcohol insoluble coating and the size of the oral dosage form. In a preferred embodiment, for a tablet form, the alcohol insoluble coating is from 5 microns to 1000 microns thick. In more preferred embodiments, for a tablet form, the alcohol insoluble coating is 5 microns to 800 microns thick, 200 microns to 1000 microns thick, 500 microns to 1000 microns thick, 200 microns to 800 microns thick, 300 microns to 700 microns thick, 400 microns to 600 microns thick, or 500 microns to 600 microns thick. In a preferred embodiment, for a pellet form, the alcohol insoluble coating is from 5 microns to 2000 microns thick. In more preferred embodiments, for a pellet form, the alcohol insoluble coating is 10 microns to 2000 microns thick, 5 microns to 1000 microns thick, 200 microns to 1500 microns thick, 200 microns to 1000 microns thick, 300 microns to 700 microns thick, 400 microns to 600 microns thick, or 500 microns to 600 microns thick.

The average transit time for an oral dosage form to move through the stomach to the intestine is 30-60 minutes, and pH 1.2 reflects the pH of the stomach, so the formulations of the invention are able to move through the stomach to the intestine in the presence of alcohol without dose dumping of the therapeutic agent. As a result, the therapeutic agent can be released as desired in the intestine.

In various other preferred embodiments, the oral dosage form of the invention releases between 0 and 35% of the therapeutic agent in vitro after 60 minutes in the presence of one or more of a variety of alcohol concentrations (such as 2%, 4%, 6%, 8%,10%,12%,14%,16%,18%,20%,22%,24%,26%,28%,30%,32%,34%,36%, 38%,40%,42%, 44%, 46%,48%,50%) at pH 1.2.

In a preferred embodiment, the therapeutic agent is an analgesic; in a more preferred embodiment the therapeutic agent is an opioid analgesic. In further preferred embodiments, the opioid analgesic is selected from the group consisting of alfentanil, allylprodine, alphaprodine, anileridine, benzylmorphine, bezitramide, buprenorphine, butorphanol, clonitazene, codeine, cyclazocine, desomorphine, dextromoramide, dezocine, diampromide, dihydrocodeine, dihydroetorphine, dihydromorphine, dimenoxadol, dimepheptanol, dimethylthiambutene, dioxaphetyl butyrate, dipipanone, eptazocine, ethoheptazine, ethylmethylthiambutene, ethylmorphine, etonitazene, etorphine, fentanyl, heroin, hydrocodone, hydromorphone, hydroxypethidine, isomethadone, ketobemidone, levallorphan, levorphanol, levophenacylmorphan, lofentanil, meperidine, meptazinol, metazocine, methadone, metopon, morphine, myrophine, nalbuphine, narceine, nicomorphine, norlevorphanol, normethadone, nalorphine, normorphine, norpipanone, opium, oxycodone, oxymorphone, papaveretum, pentazocine, phenadoxone, phenazocine, phenomorphan, phenoperidine, piminodine, piritramide, propheptazine, promedol, properidine, propiram, propoxyphene, sufentanil, tramadol, tilidine, derivatives or complexes thereof, salts thereof and combinations thereof. More preferably, the opioid analgesic is selected from the group consisting of hydrocodone, hydromorphone, oxycodone, dihydrocodeine, codeine, dihydromorphine, morphine, buprenorphine, derivatives or complexes thereof, pharmaceutically acceptable salts thereof and combinations thereof. Most preferably, the opioid analgesic is morphine, oxycodone or hydrocodone. The oral dosage forms of the present invention can accommodate a wide range of dosages of the opioid analgesic.

Pharmaceutically acceptable salts of opioid analgesics include, but are not limited to, metal salts, such as sodium salt, potassium salt, cesium salt and the like; alkaline earth metals, such as calcium salt, magnesium salt and the like; organic amine salts, such as triethylamine salt, pyridine salt, picoline salt, ethanolamine salt, triethanolamine salt, dicyclohexylamine salt, N,N′-dibenzylethylenediamine salt and the like; inorganic acid salts, such as hydrochloride, hydrobromide, sulfate, phosphate and the like; organic acid salts, such as formate, acetate, trifluoroacetate, maleate, tartrate and the like; sulfonates, such as methanesulfonate, benzenesulfonate, p-toluenesulfonate, and the like; and amino acid salts, such as arginate, asparginate, glutamate and the like.

In further embodiments, more than one opioid analgesic is included and/or a non-opioid drug is included. Such non-opioid drugs preferably provide analgesia, and include, for example, aspirin, acetaminophen, non-steroidal anti-inflammatory drugs (“NSAIDs”), N-methyl-D-aspartate (“NMDA”) receptor antagonists, cyclooxygenase-II inhibitors (“COX-II inhibitors”), and glycine receptor antagonists.

The oral dosage forms may also contain one or more aversive agents, to reduce the potential for abuse of the oral dosage form. Such aversive agents include, but are not limited to, opioid receptor antagonists (including, but not limited to, naltrexone and naloxone), bittering agents, emetics, dyes, irritants, gelling agents, and the like.

Exemplary NSAIDs include ibuprofen, diclofenac, naproxen, benoxaprofen, flurbiprofen, fenoprofen, flubufen, ketoprofen, indoprofen, piroprofen, carprofen, oxaprozin, pramoprofen, muroprofen, trioxaprofen, suprofen, aminoprofen, tiaprofenic acid, fluprofen, bucloxic acid, indomethacin, sulindac, tolmetin, zomepirac, tiopinac, zidometacin, acemetacin, fentiazac, clidanac, oxpinac, mefenamic acid, meclofenamic acid, flufenamic acid, niflumic acid, tolfenamic acid, diflurisal, flufenisal, piroxicam, sudoxicam or isoxicam, and the like. Useful dosages of these drugs are well known.

Exemplary NMDA receptor antagonists include morphinans, such as dextromethorphan or dextrophan, ketamine, d-methadone, and pharmaceutically acceptable salts thereof, and encompasses drugs that block a major intracellular consequence of NMDA-receptor activation, e.g., a ganglioside such as (6-aminothexyl)-5-chloro-1-naphthalenesulfona-mide. These drugs are stated to inhibit the development of tolerance to and/or dependence on addictive drugs, e.g., narcotic analgesics such as morphine, codeine, etc., in U.S. Pat. Nos. 5,321,012 and 5,556,838 (both to Mayer et al.), and to treat chronic pain in U.S. Pat. No. 5,502,058 (Mayer et al.), and are incorporated herein by reference. The NMDA agonist can be included alone or in combination with a local anesthetic such as lidocaine, as described in these Mayer et al. patents.

COX-II inhibitors have been reported in the art and many chemical compounds are known to produce inhibition of cyclooxygenase-II. COX-II inhibitors are described, for example, in U.S. Pat. Nos. 5,616,601; 5,604,260; 5,593,994; 5,550,142; 5,536,752; 5,521,213; 5,475,995; 5,639,780; 5,604,253; 5,552,422, 5,510,368; 5,436,265; 5,409,944 and 5,130,311, and are incorporated herein by reference. Certain preferred COX-I1 inhibitors include celecoxib (SC-58635), DUP-697, flosulide (CGP-28238), meloxicam, 6-methoxy-2-naphthylacetic acid (6-NMA), MK-966 (also known as Vioxx), nabumetone (prodrug for 6-MNA), nimesulide, NS-398, SC-5766, SC-58215, T-614, or combinations thereof. Dosage levels of COX-II inhibitor on the order of from about 0.005 mg to about 140 mg per kilogram of body weight per day have been shown to be therapeutically effective in combination with an opioid analgesic. Alternatively, about 0.25 mg to about 7 g per patient per day of a COX-II inhibitor can be administered in combination with an opioid analgesic.

The oral dosage forms of the invention may optionally contain pharmacologically inactive ingredients, i.e., pharmaceutically acceptable excipients such as polymers, suspending agents, surfactants, disintegrants, dissolution modulating components, binders, diluents, lubricants, stabilizers, antioxidants, osmotic agents, colorants, plasticizers, coatings and the like, that are used to manufacture and deliver active pharmaceutical agents. Such pharmaceutical excipients are generally incorporated into solid dosage forms to ease the manufacturing process as well as to improve the performance of the dosage form.

Such pharmacologically inactive ingredients may include the agents listed in Table 1, but are not so limited. Furthermore, the agents may be incorporated for their conventional or accepted uses in accordance with the pharmaceutical arts (for example, talc may be used as a glidant), or for non-conventional uses or to serve a different function than their accepted uses (for example, talc may be used for a reason other than to serve as a glidant, such as for use as a layering or coating agent).

Diluents, or fillers, are added in order to increase the mass of an individual dose to a size suitable for tablet compression. Preferably, such diluents are alcohol insoluble, and include but are not limited to microcrystalline cellulose and powdered cellulose. Such diluents or fillers may be used conventionally, or may be used to serve a different function, for example as a layering or coating agent.

Lubricants are incorporated into a formulation for a variety of reasons. They reduce friction between the granulation and die wall during compression and ejection. This prevents the granulate from sticking to the tablet punches, facilitates its ejection from the tablet punches, etc. Preferably, such lubricants are alcohol insoluble, such as talc, calcium stearate, and magnesium stearate. Such lubricants may be used conventionally, or may be used to serve a different function, for example as a layering or coating agent.

Glidants improve the flow characteristics of the granulation. Preferably, such glidants are alcohol insoluble, such as talc. Such glidants may be used conventionally, or may be used to serve a different function, for example as a layering or coating agent.

Binders are typically utilized if the manufacture of the dosage form uses a granulation step. Preferably, such binders are alcohol insoluble, such as include,, carboxymethylcellulose calcium, carboxymethylcellulose sodium, hydroxypropylmethylcellulose, HPMCP, and gelatin. Such binders may be used conventionally, or may be used to serve a different function, for example as a layering or coating agent.

In another aspect, the present invention provides methods for alleviating pain, comprising administering one or more oral dosage forms of the present invention to a patient in need thereof. In a preferred embodiment, the patient is one that drinks alcoholic beverages or that may be otherwise exposed to alcohol. Acceptable dosages of the opioid analgesics can be determined by a physician in light of all relevant patient information.

TABLE 1
List of ingredients that can be used to coat or cover the solid dosage formulations by layering to prevent
the dose dumping of active ingredients from solid oral dosage formulations when taken with alcohol.

| Name of Ingredients | Water | Alcohol | Comments |
|---|---|---|---|
| Calcium carbonate | I | I | By layering in combination with binder and/or |
| | | | other ingredients |
| Dibasic/Tribasic calcium phosphate | I | I | By layering in combination with binder and/or |
| dihydrate | | | other ingredients |
| Calcium Stearate | I | I | By layering in combination with binder and/or |
| | | | other ingredients |
| Calcium Sulfate | I | I | By layering in combination with binder and/or |
| | | | other ingredients |
| Carboxymethylcellulose calcium | I | I | By layering in combination with binder and/or |
| | | | other ingredients |
| Carboxymethylcellulose Sodium | I | I | By coating alone or in combination with binder and/ |
| | | | or other ingredients |
| Microcrystalline Cellulose | I | I | By layering in combination with binder and/or |
| | | | other ingredients |
| Powdered Cellulose | I | I | By layering in combination with binder and/or |
| | | | other ingredients |
| CAP | I | I | Enteric coating agent. By coating alone or in |
| | | | combination with binder and/or other ingredients |
| Gelatin | S | I | Coating agent. By coating alone or in combination |
| | | | with binder and/or other ingredients |
| Glyceryl Palmitate | I | I | By layering in combination with binder and/or |
| | | | other ingredients |
| Glyceryl Behanate | I | I | By layering in combination with binder and/or |
| | | | other ingredients |
| Glyceryl Monostearate | I | I | By layering in combination with binder and/or |
| | | | other ingredients |
| Hydroxyethyl Cellulose (HEC) | S | I | Coating polymer. By coating alone or in |
| | | | combination with binder and/or other ingredients |
| Hdroxypropylmethylcellulose (HPMC) | S | I | Coating polymer. By coating alone or in |
| | | | combination with binder and/or other ingredients |
| HPMCP | I | I | Enteric coating polymer. By coating alone or in |
| | | | combination with binder and/or other ingredients |
| Kaolin | I | I | By layering in combination with binder and/or |
| | | | other ingredients |
| Mg-Aluminium Silicate | I | I | By layering in combination with binder and/or |
| | | | other ingredients |
| Magnesium carbonate | I | I | By layering in combination with binder and/or |
| | | | other ingredients |
| Magnesium Oxide | I | I | By layering in combination with binder and/or |
| | | | other ingredients |
| Magnesium Stearate | I | I | By layering in combination with binder and/or |
| | | | other ingredients |
| Magnesium Trisilicate | I | I | By layering in combination with binder and/or |
| | | | other ingredients |
| Methyl Cellulose | Partially | I | Coating polymer. By coating alone or in |
| | soluble | | combination with binder and/or other ingredients |
| Colloidal Silicon Dioxide | I | I | By layering in combination with binder and/or |
| | | | other ingredients |
| Talc | I | I | By layering in combination with binder and/or |
| | | | other ingredients |
| Carnuba wax | I | I | Coating agent. By coating alone or in combination |
| | | | with binder and/or other ingredients |
| Zein | I | I | By coating alone or in combination with binder and/ |
| | | | or other ingredients |

S = Soluble,
I = Insoluble
1. The major claim of the invention is the use of enteric polymers insoluble in water and alcohol e.g., like CAP, HPMCP, EC (containing Ethoxyl groups less than 46.5%).
2. The invention covers the coating or layering of drug substance and/or drug product.
3. The invention covers the products where osmotic push pull system is used as a drug delivery technology and one of the above polymers are used in coating for the intended use
4. The invention also covers for the hard gel and soft gel capsules where one of the components of the capsule shell is in the above list (excluding gelatin).
5. the invention also covers the use of alcohol insoluble plasticizers along with the above mentioned coating agents
6. This invention covers all modified release solid dosage formulations

I claim:

1. A modified release oral dosage form, comprising
(a) a therapeutic agent and;
(b) an alcohol insoluble coating,
wherein between 0% and 35% of the therapeutic agent is released from the dosage form in vitro after 60 minutes in the presence of 40% alcohol at pH 1.2.

2. The modified release oral dosage form of claim 1, wherein between 0% and 20% of the therapeutic agent is released from the dosage form in vitro after 60 minutes in the presence of 40% alcohol at pH 1.2.

3. The modified release oral dosage form of claim 1, wherein between 0% and 10% of the therapeutic agent is released from the dosage form in vitro after 60 minutes in the presence of 40% alcohol at pH 1.2.

4. The modified release oral dosage form of claim 1, wherein the modified release comprises extended release.

5. The modified release oral dosage form of claim 1, wherein the modified release comprises delayed release.

6. The modified release oral dosage form of claim 1, wherein the alcohol insoluble coating is water insoluble.

7. The modified release oral dosage form of claim 1, wherein the alcohol insoluble coating comprises one or more compounds listed in Table 1.

8. The modified release oral dosage form of claim 1, wherein the modified release oral dosage comprises a tablet.

9. The modified release oral dosage form of claim 1, wherein the modified release oral dosage comprises a capsule.

10. The modified release oral dosage form of claim 1, wherein the alcohol insoluble coating comprises a 1% to 40% weight gain in the oral dosage form.

11. The modified release oral dosage form of claim 1, wherein the alcohol insoluble coating is between 5 microns thick and 1000 microns thick.

12. The modified release oral dosage form of claim 1, wherein the therapeutic comprises an analgesic, or a pharmaceutically acceptable salt thereof.

13. The modified release oral dosage form of claim 1, wherein the analgesic comprises an opioid analgesic, or a pharmaceutically acceptable salt thereof.

14. The modified release oral dosage form of claim 13, wherein the therapeutic further comprises a non-opioid drug.

15. A method for alleviating pain, comprising administering to an individual in need thereof an amount effective to alleviate pain of the oral dosage form of claim 12.

16. A method for alleviating pain, comprising administering to an individual in need thereof an amount effective to alleviate pain of the oral dosage form of claim 13.

17. A method for alleviating pain, comprising administering to an individual in need thereof an amount effective to alleviate pain of the oral dosage form of claim 14.

18. The method of claim 15, wherein the individual in need thereof drinks alcoholic beverages or is otherwise exposed to alcohol.

19. The method of claim 16, wherein the individual in need thereof drinks alcoholic beverages or is otherwise exposed to alcohol.

20. The method of claim 17, wherein the individual in need thereof drinks alcoholic beverages or is otherwise exposed to alcohol.
